(19) United States
(12) Patent Application Publication
(10) Pub. No.: US 2010/0291116 A1
(43) Pub. Date: Nov. 18, 2010

(54) MICROPARTICLE COMPRISING CROSS-LINKED POLYMER

(75) Inventors: Aylvin Jorge Angelo Athanasius Dias, Maastricht (NL); Bartholomeus Johannes Margretha Plum, Ulestraten (NL); Audrey Petit, Maastricht (NL); Tristan Handels, Beek (NL)

Correspondence Address: NIXON & VANDERHYE, PC, 901 NORTH GLEBE ROAD, 11TH FLOOR, ARLINGTON, VA, 22203, US

(73) Assignee: DSM IP ASSETS B.V., HEERLEN (NL)

(21) Appl. No.: 12/679,132

(22) PCT Filed: Sep. 26, 2008
(86) PCT No.: PCT/EP08/62981

§ 371 Date: Jul. 26, 2010

(30) Foreign Application Priority Data
Sep. 26, 2007 (EP) 070189.0.3

(51) Int. Cl. A61K 47/30; C08G 67/00; B32B 1/00; A61K 38/02; A61K 39/00; A61K 31/7088; C08G 73/00; C08G 75/00; A61K 31/573; A61P 31/00

(52) U.S. Cl. 424/184.1; 514/772.3; 528/271; 428/402; 428/407; 514/1.1; 514/44 R; 528/367; 528/390; 528/363; 514/180

(57) ABSTRACT

Microparticle comprising a cross-linked polymer comprising (a) a cross-linker comprising two or more radically polymerizable groups, preferably selected from the group consisting of alkenes, sulfhydryl (SH), thioic, unsaturated esters, unsaturated urethanes, unsaturated ethers, and unsaturated amides; (b) a monofunctional reactive diluent comprising maximum one unsaturated C—C bond represented by the formula R₀—C(R₁)═CHR₂ Formula (I) wherein —R₀ is chosen depending on the structure of a selected active agent (c) to be loaded into the microparticle and is chosen to have a structure that when combined with the other components of the microparticle provides a higher affinity of the selected active agent (c) for the microparticle; —each R₁ is chosen from hydrogen and substituted and unsubstituted, aliphatic, cycloaliphatic and aromatic hydrocarbon groups which groups optionally contain one or more moieties selected from the group of ester moieties, ether moieties, thioester moieties, thioether moieties, carbamate moieties, thiocarbamate moieties, amide moieties and other moieties comprising one or more heteroatoms, in particular one or more heteroatoms selected from S, O, P and N, each R₅ in particular independently being chosen from the group of hydrogen and substituted and unsubstituted alkyl groups, which alkyl groups optionally contain one or more heteroatoms, in particular one or more heteroatoms selected from P, S, O and N; —each R₂ is chosen from hydrogen, —COOCH₃, —COOC₂H₅, —COOC₃H₇, and —COOC₄H₉.

The invention relates to a microparticle comprising a cross-linked polymer, a method for preparing such microparticle, and the use of said microparticle in medical applications.

Spherical microparticles (microspheres) comprising cross-linked polymers are described in WO 98/22093. These microspheres are intended for use as a delivery system for a releasable compound (a drug). It is stated that the cross-linkable polymer used to prepare the particles is not critical. Suitable polymers mentioned in this publication are cross-linkable water-soluble dextrans, derivatized dextrans, starches, starch derivatives, cellulose, polyvinylpyrrolidone, proteins and derivatized proteins.

A disadvantage of the above mentioned microparticles is that the pore size of the cross-linked polymer must be smaller than the particle size of the releasable compound. Thus, it is not possible to load the microspheres with the releasable compound after the microspheres have been made. It is therefore not possible to prepare a master batch of the microspheres without the releasable compound and to decide later which releasable compound to include in the microspheres. A further disadvantage is that it is very difficult to tune the release of drugs. For particular applications a faster or slower release of a particular drug may be required.

It would however be desirable to be able to load microparticles afterwards, because it would allow one to target and separate a desired microparticle size for subsequent loading with an active agent. In addition it would be possible to upscale the microspheres that would follow a masterbatch production strategy for active agents and—if desired—different portions can be loaded with different active agents, in useful quantities for a specific purpose. Furthermore, it would be desirable to be able to load microparticles after their formation in case an agent to be released from the microparticles is detrimentally affected, e.g. degraded, denaturated or otherwise inactivated, during the preparation of the microparticles. This is particularly the case for active agents thermally sensitive, photo or irradiation sensitive and sensitive to the reactive groups that form the microparticle directly or indirectly.

There is a continuous need for alternative or improved microparticles comprising a cross-linked polymer that can be adequately loaded with an active agent, such as enzymes, proteins and small molecule drugs after the microparticle has been prepared. It would be more desirable to be able to tune release of the active agent in the microparticles. It would be more desirable to provide microparticles with a different loading capacity for the selected active agent.

Accordingly, it is an object of the present invention to provide a novel microparticle that can serve at least as an alternative to known microparticles and in particular to provide a microparticle that is effectively loadable with an active agent.

Another object of the present invention is to provide a microparticle having one or more other favourable properties as identified herein below.

According to the present invention it has been found to provide a microparticle comprising a cross-linked polymer suitable for loading with a selective active agent comprising
(a) a cross-linker comprising two or more radically polymerizable groups, preferably selected from the group consisting of alkenes, sulfhydryl (SH), thioic acids, unsaturated esters, unsaturated urethanes, unsaturated ethers, and unsaturated amides; and
(b) a monofunctional reactive diluent comprising maximum one unsaturated C—C bond represented by the formula

R₀—C(R₁)═CHR₂ Formula I

wherein

R₀ is chosen depending on the structure of a selected active agent (c) to be loaded into the microparticle and is chosen to have a structure that when combined with the other components of the microparticle provides a higher affinity of the selected active agent (c) for the microparticle;

each R₁ is chosen from hydrogen and substituted and unsubstituted, aliphatic, cycloaliphatic and aromatic hydrocarbon groups which groups optionally contain one or more moieties selected from the group of ester moieties, ether moieties, thioester moieties, thioether moieties, carbamate moieties, thiocarbamate moieties, amide moieties and other moieties comprising one or more heteroatom chosen from S, O, P and N,

each R₂ is chosen from hydrogen, —COOCH₃, —COOC₂H₅, —COOC₃H₇, and —OOC₄H₉.

It has surprisingly been found that the use of cross-linker (a) in combination with reactive diluent (b) results in microparticles with a different loading capacity for the selected active agent (c). As such the release of the active agent can be tuned or altered without the use of a different cross-linker.

A reactive diluent as used in the present invention means a monofunctional diluent with comprises maximum one unsaturated bond.

Suitable examples of R₀ are functional groups that are linear, (hyper)branched or cyclic. These structures may possess a hetero atom, for example O, N, S, or P. The linear and (hyper)branched R₀ groups may comprise amine, amide, carbamate, urea, thiol, hydroxyl, carboxyl, ester, ether, thioester, thioester carbonate, phosphate, posphite, sulphate, sulphoxide and/or sulphone groups.

Suitable examples of cyclic R₀ groups include aromatic and cyclic aliphatic groups. Suitable examples of heterocyclic R₀ groups include 5-membered ring phosphate, 6-membered ring phosphate, 5-membered ring phosphite, 6-membered ring phosphite, 4-membered ring lacton, 5-membered ring lacton, 6-membered ring lacton, 5-membered ring carbonate, 6-membered ring carbonate, 5-membered ring sulphate, 6-membered ring sulphate, 5 ring sulphoxide, 6-membered ring sulphoxide, 6-membered ring amide, 5-membered ring urethane, 6-membered ring urethane, 7-membered ring urethane, 5-membered ring urea, 6-membered ring urea, and 7-membered ring urea.

Preferred are components that have a urethane group in the molecule and a 5-membered ring phosphate, 6-membered ring phosphate, 5-membered ring phosphite, 6-membered ring phosphite 4 ring lacton, 5-membered ring lacton, 6-membered ring lacton, 5-membered ring carbonate, 6-membered ring carbonate, 5-membered ring sulphate, 6-membered ring sulphate, 5 ring sulphoxide, 6-membered ring sulphoxide, 5-membered ring amide, 6-membered ring amide, 7 ring amide, 5-membered ring urethane, 6-membered ring urethane, 7-membered ring urethane, 5-membered ring urea, 6-membered ring urea, 7-membered ring urea group.

Also very reactive and preferred components are components having both a carbonate functionality in the molecule and a functionality selected from the list consisting of a 5 ring phosphate, 6-membered ring phosphate, 5-membered ring phosphite, 6-membered ring phosphite, 4-membered ring lacton, 5-membered ring lacton, 6-membered ring lacton, 5-membered ring carbonate, 6-membered ring carbonate, 5-membered ring sulphate or sulphite, 6-membered ring sulphate or sulphite, 5-membered ring sulphite, 6-membered ring sulphite, 5 ring sulphoxide, 6-membered ring sulphoxide, 5-membered ring amide, 5-membered ring imide, 6-membered ring amide, 7 ring amide, 5-membered ring imide, 6-membered ring imide, 5-membered ring thioimide, 6-membered ring thioimide, 5-membered ring urethane, 6-membered ring urethane, 7-membered ring urethane, 5-membered ring urea, 6-membered ring urea and 7-membered ring urea group.

R₁ is independently chosen from the group of hydrogen and substituted or unsubstituted alkyl groups, which alkyl groups optionally contain one or more heteroatoms chosen from P, S, O and N. Preferably R₁ is chosen from hydrogen or a hydrocarbon comprising up to 12 carbons. In particular R₁ may be hydrogen or a substituted or unsubstituted C₁ to C₆ alkyl, more in particular a substituted or unsubstituted C₁ to C₃ alkyl. Optionally R₁ comprises a carbon-carbon double or triple bond, in particular R₁ may comprise a —CH═CH₂ group.

R₂ is preferably hydrogen.

Suitable reactive diluents (b) include acrylic compounds or other olefinically unsaturated compounds, for example, vinyl ether, allylether, allylurethane, fumarate, maleate, itaconate or unsaturated (meth)acrylate units. Suitable unsaturated (meth)acrylates are, for example, unsaturated urethane(meth)acrylates, unsaturated polyester(meth)acrylates, unsaturated epoxy(meth)acrylates and unsaturated polyether(meth)acrylates.

Particularly suitable examples of reactive diluents (b) with linear, (hyper)branched or cyclic R₀ groups are listed in Table 1.

TABLE 1
Examples of reactive diluent (b)

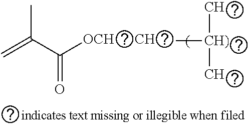

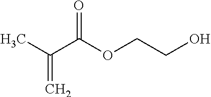

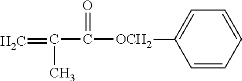

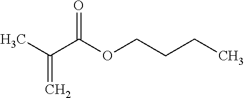

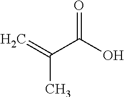

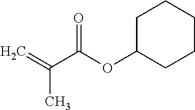

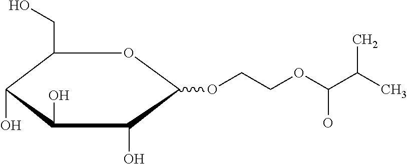

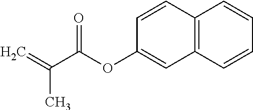

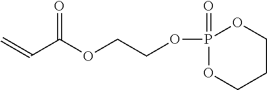

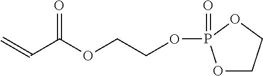

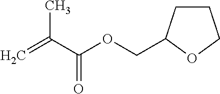

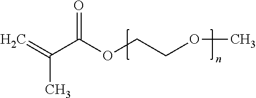

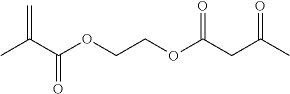

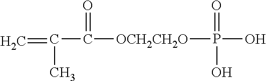

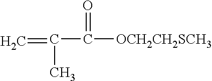

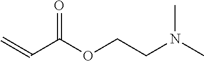

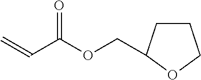

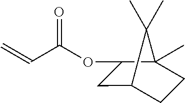

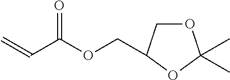

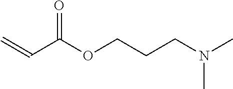

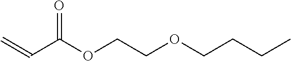

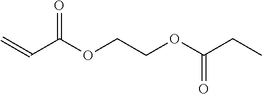

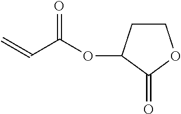

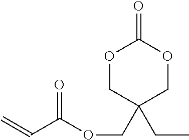

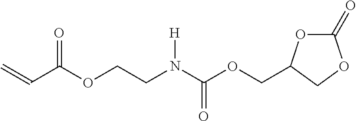

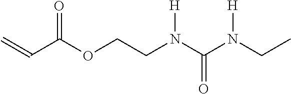

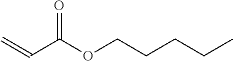

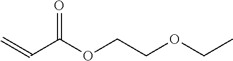

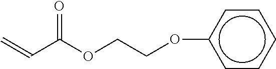

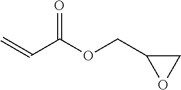

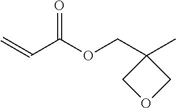

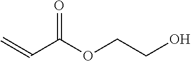

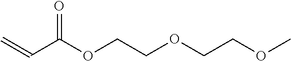

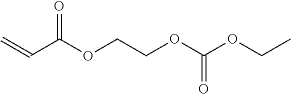

In particular cross-linker (a) comprises two or more —CR₃═CHR₄ groups wherein

each R₃ is independently chosen from hydrogen and substituted and unsubstituted, aliphatic, cycloaliphatic and aromatic hydrocarbon groups which groups optionally contain one or more moieties selected from the group of ester moieties, ether moieties, thioester moieties, thioether moieties, carbamate moieties, thiocarbamate moieties, amide moieties and other moieties comprising one or more heteroatoms, in particular one or more heteroatoms selected from S, O, P and N, each R₃ in particular independently being chosen from the group of hydrogen and substituted and unsubstituted alkyl groups, which alkyl groups optionally contain one or more heteroatoms, in particular one or more heteroatoms selected from P, S, O and N;

each R₄ is chosen from hydrogen, —COOCH₃, —COOC₂H₅, —COOC₃H₇, —COOC₄H₉.

Even more in particular cross-linker (a) is a compound with formula

X—[Y—C(═Z)—N(R₅)—R₆—C(R₃)═CR₄]ₙ Formula II

wherein

X is a residue of a multifunctional radically polymerisable compound (having at least a functionality equal to n);

each Y independently is optionally present, and—if present—each Y independently represents a moiety selected from the group of O, S and NR₅;

each Z is independently chosen from O and S;

each R₃ and R4 are as defined above;

each R₅ is independently chosen from the group of hydrogen and substituted and unsubstituted, aliphatic, cycloaliphatic and aromatic hydrocarbon groups which groups optionally contain one or more moieties selected from the group of ester moieties, ether moieties, thioester moieties, thioether moieties, carbamate moieties, thiocarbamate moieties, amide moieties and other moieties comprising one or more heteroatoms, in particular one or more heteroatoms selected from S, O, P and N,

each R₆ is independently chosen from the group of substituted and unsubstituted, aliphatic, cycloaliphatic and aromatic hydrocarbon groups which groups optionally contain one or more moieties selected from the group of ester moieties, ether moieties, thioester moieties, thioether moieties, carbamate moieties, thiocarbamate moieties, amide moieties and other moieties comprising one or more heteroatoms, in particular one or more heteroatoms selected from S, O, P and N; and

n is at least 2.

R₅ is in particular independently chosen from the group of hydrogen and substituted and unsubstituted alkyl groups, which alkyl groups optionally contain one or more heteroatoms, in particular one or more heteroatoms selected from P, S, O and N. In more particular R₅ is hydrogen or a hydrocarbon comprising up to 12 carbons. R₅ may be hydrogen or a substituted or unsubstituted C₁ to C₆ alkyl. R₅ may also be a substituted or unsubstituted cycloalkyl, more in particular a substituted or unsubstituted C₁ to C₃ alkyl or hydrogen. The cycloalkyl may be a cyclopentyl, cyclohexyl or cycloheptyl. The alkyl may be a linear or branched alkyl. A preferred branched alkyl is t-butyl. Optionally R₅ may comprise a carbon-carbon double or triple bond, R₅ may for example comprise a —CH═CH₂ group. R₅ may comprise an heteroatom, for example an ester moiety, such as —(C═O)—O—(CH₂)ᵢ—CH₃ or —(C═O)—O—(CH₂)ᵢ—CH═CH₂, wherein i is an integer, usually in the range of 0-8, preferably in the range of 1-6. The heteroatom may also be a keto-moiety, such as. —(C═O)—(CH₂)ᵢ—CH₃ or —(C═O)—(CH₂)ᵢ—CH═CH₂, wherein i is an integer, usually in the range of 0-8, preferably in the range of 1-6. An R₅ group comprising a heteroatom preferably comprises a NR′R″ group, wherein R′ and R″ are independently a hydrogen or a hydrocarbon group, in particular a C1-C6 alkyl. More preferred R₅ is hydrogen or an alkyl group. Still more preferably, R₅ is hydrogen or a methyl group.

R₆ preferably comprises 1-20 carbon atoms. More preferably R₆ is a substituted or unsubstituted C₁ to C₂₀ alkylene, in particular a substituted or unsubstituted C₂ to C₁₄ alkylene. R₆ may comprise an aromatic moiety, such as o-phenylene, m-phenylene or p-phenylene. The aromatic moiety may be unsubstituted or substituted, for instance with an amide, for example an acetamide.

R₆ may comprise a —(O—C═O)—, a —(N—C═O), a —(O—C═S)— functionality. It is also possible that R₆ comprises an alicyclic moiety, for example a cyclopentylene, cyclohexylene or a cycloheptylene moiety, which optionally comprises one or more heteroatoms for example a N-group and/or a keto-group.

Optionally R₆ comprises a carbon-carbon double or triple bond, in particular R₆ may comprise a —CH═CH₂ group. In a preferred embodiment R₆ is chosen from a —CH₂—CH₂—O—C(O)—, —CH₂—CH₂—N—C(O)— or —CH₂—CH₂—O—C(S)— group.

R₃ is for example hydrogen or a hydrocarbon comprising up to 12 carbons. In particular R₃ may be hydrogen or a substituted or unsubstituted C₁ to C₆ alkyl, more in particular a substituted or unsubstituted C₁ to C₃ alkyl.

Optionally R₃ comprises a carbon-carbon double or triple bond, in particular R₃ may comprise a —CH═CH₂ group.

R₄ is preferably hydrogen.

n is preferably 2-8.

Substituents on R₅, R₆ and/or R₃ may for example be chosen from halogen atoms and hydroxyl. A preferred substituent is hydroxyl. In particular R₆ is a —CH₂OH group because it is commercially available.

The polymer is generally cross-linked via reaction of vinylic bonds of the cross-linker.

Advantageously, the microparticle, which may be a microsphere, in particular in case if the cross-linked polymer is a carbamate, thiocarbamate, a ureyl or an amide copolymer, is tough but still elastic. This is considered beneficial with respect to allowing processing under aggressive conditions, such as sudden pressure changes, high temperatures, low temperatures and/or conditions involving high shear.

The microparticles of the present invention show a good resistance against a sudden decrease in temperature, which may for example occur if the microparticles are lyophilised.

In a preferred embodiment, the microparticles according to the present invention are even essentially free of cryoprotectants. A cryoprotectant is a substance that protects a material, i.c. microparticles, from freezing damage (damage due to ice formation). Examples of cryoprotectants include a glycol, such as ethylene glycol, propylene glycol and glycerol or dimethyl sulfoxide (DMSO).

It is further envisaged that the microparticles of the present invention show a good resistance against heating, which may occur if the particles are sterilised (at temperatures above 120° C.) or if the particles are loaded with an active substance at elevated temperatures for example temperatures above 100° C.

The microparticles of the present invention may be used in medical applications such as a delivery system for an active agent, in particular a drug, a diagnostic aid or an imaging aid. The microparticles can also be used to fill a capsule or tube by using high pressure or may be compressed as a pellet, without substantially damaging the microparticles. It can also be used in injectable or spray-able form as a suspension in a free form or in an in-situ forming gel formulation. Furthermore, the microparticles can be incorporated in for example (rapid prototyped) scaffolds, coatings, patches, composite materials, gels or plasters.

The microparticle according to the present invention can be injected, sprayed, implanted or absorbed.

Y in formula II is optionally present, and—if present—each Y independently represents a moiety selected from the group of O, S and NR₅.

X in formula II is a residue of a multifunctional radically polymerisable compound, preferably X is a residue of a —OH, —NH₂, —RNH or —SH multifunctional polymer or oligomer. The multifunctional polymer or oligomer is in particular selected from biostable or biodegradable polymers or oligomers that can be natural or synthetic.

The term biodegradable refers to materials that experience degradation by hydrolysis or by the action of an enzyme or by the action of biological agents present in their environment such as bacteria and fungi. Such may be attributable to a microorganism and/or it may occur in the body of an animal or a human.

The term biostable refers to materials which are not substantially broken down in a biological environment, in case of an implant at least not noticeably within a typical life span of a subject, in particular a human, wherein the implant has been implanted.

Examples of biodegradable polymers are polylactide (PLA); polyglycolide (PGA), polydioxanone, poly(lactide-co-glycolide), poly(glycolide-co-polydioxanone), polyanhydrides, poly(glycolide-co-trimethylene carbonate), poly(glycolide-co-caprolactone), poly-(trimethylenecarbonates), aliphatic polyesters, poly(orthoesters); poly(hydroxyl-acids), polyamino-carbonates or poly(ε-caprolactones) (PCL).

Examples of biostable or synthetic polymers are poly(urethanes); poly(vinyl alcohols) (PVA); polyethers, such as poly alkylene glycols, preferably poly (ethylene glycols) (PEG); polythioethers, aromatic polyesters, aromatic thioesters, polyalkylene oxides, preferably selected from poly(ethylene oxides) and poly (propylene oxides); poloxamers, meroxapols, poloxamines, polycarbonates, poly(vinyl pyrrolidones): poly(ethyl oxazolines).

Examples of natural polymers are polypeptides, polysaccharides for example polysucrose, hyaluronic acid, dextran and derivates thereof, heparin sulfate, chondroitin sulfate, heparin, alginate, and proteins such as gelatin, collagen, albumin, ovalbumin, starch, carboxymethylcellulose or hydroxyalkylated cellulose and co-oligomers, copolymers, and blends thereof.

X in formula II may be chosen based upon its biostability/biodegradability properties. For providing microparticles with high biostability polyethers, polythioethers, aromatic polyesters or aromatic thioesters are generally particularly suitable. For providing microparticles with high biodegradability aliphatic polyesters, aliphatic polythioesters, aliphatic polyamides, aliphatic polycarbonates or polypeptides are particularly suitable. Preferably X is selected from an aliphatic polyester, aliphatic polythioester, aliphatic polythioether, aliphatic polyether or polypeptide. More preferred are copolymersor blends comprising PLA, PGA, PLGA, PCL and/or poly(ethylene oxide)-co-poly(propylene oxide) block co-oligomers/copolymers.

A combination of two or more different moieties forming X may be used to adapt the degradation rate of the particles and/or the release rate of an active agent loaded in or on the particles, without having to change the particle size, although of course one may vary the particle size, if desired. The two or more different moieties forming X are for example a copolymer or co-oligomer (i.e. a polymer respectively oligomer comprising two or more different monomeric residues). A combination of two or more different moieties forming X may further be used to alter the loading capacity, change a mechanical property and/or the hydrophilicity/hydrophobicity of the microparticles.

The (number average) molecular weight of the X-moiety is usually chosen in the range of 100 to 100,000 g/mol. In particular, the (number average) molecular weight may be at least 200, at least 500, at least 700 or at least 1000 g/mol. In particular, the (number average) molecular weight may be up to 50,000 or up to 10 000 g/mol. In the present invention the (number average) molecular weight is as determinable by size exclusion chromatography (GPC), using the method as described in the Examples.

In a preferred embodiment, the X-moiety in the cross-linked polymer is based on a compound having at least two functionalities that can react with an isocyanate to form a carbamate, thiocarbamate or ureyl link. In such an embodiment, the Y group is present in formula I. The X moiety is usually a polymeric or oligomeric compound with a minimum of two reactive groups, such as hydroxyl (—OH), amine or thiol groups.

In another embodiment, X is the residue of a amine-bearing compound to provide an alkenoyl urea, providing a compound represented by the formula, X—(N—CO—NR—CO—CH═CH₂)ₙ or X—(N—CO—NR—CO—C(CH₃)═CH2)ₙ). Examples thereof are in particular poly(propenoylurea), poly(methylpropenoylurea) or poly(butenoylurea). Herein each R independently represents a hydrocarbon group such as identified above.

In still another embodiment, X is the residue of a thiol-bearing compound to provide a compound represented by the formula X—(S—C(S)—NH-Phenyl-CH═CH₂)₂, such as a poly(alkenyl carbamodithioic) ester.

In a further embodiment, X is the residue of a carboxylic acid bearing compound to provide a compound represented by the formula X—(C(O)—NR—C(O)—CH═CH2)ₙ. Herein each R independently represents a hydrocarbon group such as identified above. An example thereof is poly((methyl-)oxo-propenamide.

As used in this application, the term “oligomer” in particular means a molecule essentially consisting of a small plurality of units derived, actually or conceptually, from molecules of lower relative molecular mass. It is to be noted that a molecule is regarded as having an intermediate relative molecular mass if it has properties which vary significantly with the removal of one or a few of the units. It is also to be noted that, if a part or the whole of the molecule has an intermediate relative molecular mass and essentially comprises a small plurality of the units derived, actually or conceptually, from molecules of lower relative molecular mass, it may be described as oligomeric, or by oligomer used adjectivally. In general, oligomers have a molecular weight of more than 200 Da, such as more than 400, 800, 1000, 1200, 2000, 3000, or more than 4000 Da. The upper limit is defined by what is defined as the lower limit for the mass of polymers (see next paragraph).

Accordingly the term “polymer” denotes a structure that essentially comprises a multiple repetition of units derived, actually or conceptually, from molecules of low relative molecular mass. Such polymers may include cross-linked networks, branched polymers and linear polymers. It is to be noted that in many cases, especially for synthetic polymers, a molecule can be regarded as having a high relative molecular mass if the addition or removal of one or a few of the units has a negligible effect on the molecular properties. This statement fails in the case of certain macromolecules for which the properties may be critically dependant on fine details of the molecular structure. It is also to be noted that, if a part or the whole of the molecule has a high relative molecular mass and essentially comprises the multiple repetition of units derived, actually or conceptually, from molecules of low relative molecular mass, it may be described as either macromolecular or polymeric, or by polymer used adjectivally. In general, polymers have a molecular weight of more than 8000 Da, such as more than 10,000, 12,000, 15,000, 25,000, 40,000, 100,000 or more than 1,000,000 Da.

Microparticles have been defined and classified in various different ways depending on their specific structure, size, or composition, see e.g. Encyclopaedia of Controlled drug delivery Vol 2 M-Z Index, Chapter: Microencapsulation Wiley Interscience, starting at page 493, see in particular page 495 and 496.

As used herein, microparticles include micro- or nanoscale particles which are typically composed of solid or semi-solid materials and which are capable of carrying an active agent. Typically, the average diameter of the microparticles given by the Fraunhofer theory in volume percent ranges from 10 nm to 1000 μm. The preferred average diameter depends on the intended use. For instance, in case the microparticles are intended for use as an injectable drug delivery system, in particular as an intravascular drug delivery system, an average diameter of up to 10 μm, in particular of 1 to 10 μm may be desired.

It is envisaged that microparticles with a average diameter of less than 800 nm, in particular of 500 nm or less, are useful for intracellular purposes. For such purposes, the average diameter preferably is at least 20 nm or at least 30 nm. In other applications, larger dimensions may be desirable, for instance a diameter in the range of 1-100 μm or 10-100 μm. In particular, the particle diameter as used herein is the diameter as determinable by a LST 230 Series Laser Diffraction Particle size analyzer (Beckman Coulter), making use of a UHMW-PE (0.02-0.04 μm) as a standard. Particle-size distributions are estimated from Fraunhofer diffraction data and given in volume (%). If the particles are too small or non analyzable by light scattering because of their optical properties then scanning electron microscopy (SEM) or transmission electron microscopy (TEM) can be used.

Several types of microparticle structures can be prepared according to the present invention. These include substantially homogenous structures, including nano- and microspheres and the like. However in case that more than one active agent has to be released or in case that one or more functionalities are needed it is preferred that the microparticles are provided with a structure comprising an inner core and an outer shell. A core/shell structure enables more multiple mode of action for example in in drug delivery of incompatible compounds or in imaging. The shell can be applied after formation of the core using a spray drier. The core and the shell may comprise the same or different cross-linked polymers with different active agents. In this case it is possible to release the active agents at different rates. It is also possible that the active agent is only present in the core and that the shell is composed of cross-linked polymers capable to provide lubricity.

In a further embodiment the microparticles may comprise a core comprising the cross-linked polymers according to the present invention and a shell comprising a magnetic or magnetisable material.

In still a further embodiment, the microparticles may comprise a magnetic or magnetisable core and a shell comprising the cross-linked polymers according to the present invention. Suitable magnetic or magnetisable materials are known in the art. Such microparticles may be useful for the capability to be attracted by objects comprising metal, in particular steel, for instance an implanted object such as a graft or a stent. Such microparticles may further be useful for purification or for analytical purposes.

In a still further embodiment, the particles are imageable by a specific technique. Suitable imaging techniques are MRI, CT, X-ray. The imaging agent can be incorporated inside the particles or coupled onto their surface. Such particles may be useful to visualize how the particles migrate, for instance in the blood or in cells. A suitable imaging agent is for example gadolinium.

The microparticles according to the present invention may carry one or more active agents (c). The microparticle according to the invention is particularly suitable to be loaded with active agent (c) because it has a high loading capacity for active agent (c). The active agent (c) may be more or less homogeneously dispersed within the microparticles or within the microparticle core. The active agent (c) may also be located within the microparticle shell.

In particular, the active agent (c) may be selected from the group of nutrients, pharmaceuticals, proteins and peptides, vaccines, genetic materials, (such as polynucleotides, oligonucleotides, plasmids, DNA and RNA), diagnostic agents, and imaging agents. The active agent (c), such as an active pharmacologic ingredient (API), may demonstrate any kind of activity, depending on the intended use.

The active agent (c) may be capable of stimulating or suppressing a biological response. The active agent (c) may for example be chosen from growth factors (VEGF, FGF, MCP-1, PIGF, antibiotics (for instance penicillin's such as B-lactams, chloramphenicol), anti-inflammatory compounds, antithrombogenic compounds, anti-claudication drugs, anti-arrhythmic drugs, anti-atherosclerotic drugs, antihistamines, cancer drugs, vascular drugs, ophthalmic drugs, amino acids, vitamins, hormones, neurotransmitters, neurohormones, enzymes, signalling molecules and psychoactive medicaments.

Examples of specific active agents (c) are neurological drugs (amphetamine, methylphenidate), alpha1 adrenoceptor antagonist (prazosin, terazosin, doxazosin, ketenserin, urapidil), alpha2 blockers (arginine, nitroglycerin), hypotensive (clonidine, methyldopa, moxonidine, hydralazine minoxidil), bradykinin, angiotensin receptor blockers (benazepril, captopril, cilazepril, enalapril, fosinopril, lisinopril, perindopril, quinapril, ramipril, trandolapril, zofenopril), angiotensin-1 blockers (candesartan, eprosartan, irbesartan, losartan, telmisartan, valsartan), endopeptidase (omapatrilate), beta2 agonists (acebutolol, atenolol, bisoprolol, celiprolol, esmodol, metoprolol, nebivolol, betaxolol), beta2 blockers (carvedilol, labetalol, oxprenolol, pindolol, propanolol) diuretic actives (chlortalidon, chlorothiazide, epitizide, hydrochlorthiazide, indapamide, amiloride, triamterene), calcium channel blockers (amlodipin, barnidipin, diltiazem, felodipin, isradipin, lacidipin, lercanidipin, nicardipin, nifedipin, nimodipin, nitrendipin, verapamil), anti arthymic active (amiodarone, solatol, diclofenac, enalapril, flecamide) or ciprofloxacin, latanoprost, flucloxacillin, rapamycin and analogues and limus derivatives, paclitaxel, taxol, cyclosporine, heparin, corticosteroids (triamcinolone acetonide, dexamethasone, fluocinolone acetonide), anti-angiogenic (iRNA, VEGF antagonists: bevacizumab, ranibizumab, pegaptanib), growth factor, zinc finger transcription factor, triclosan, insulin, salbutamol, oestrogen, norcantharidin, microlidil analogues, prostaglandins, statins, chondroitinase, diketopiperazines, macrocycli compounds, neuregulins, osteopontin, alkaloids, immuno suppressants, antibodies, avidin, biotin, clonazepam.

The active agent (c) can be delivered for local delivery or as pre or post surgical therapies for the management of pain, osteomyelitis, osteosarcoma, joint infection, macular degeneration, diabetic eye, diabetes mellitus, psoriasis, ulcers, atherosclerosis, claudication, thrombosis viral infection, cancer or in the treatment of hernia.

In accordance with the present invention, if an active agent (c) is present, the concentration of one or more active agent in the microparticles, is preferably at least 5 wt. %, based on the total weight of the microparticles, in particular at least 10 wt. %, more in particular at least 20 wt. %. The concentration may be up to 90 wt. %, up to 70 wt. %, up to 50 wt. % or up to 30 wt. %, as desired.

The fields wherein microparticles according to the present invention can be used include dermatology, vascular, orthopedics, ophthalmic, spinal, intestinal, pulmonary, nasal, or auricular.

Besides in a pharmaceutical application, microparticles according to the invention may inter alia be used in an agricultural application. In particular, such microparticles may comprise a pesticide or a plant-nutrient.

It is also possible to functionalise at least the surface of the microparticles by providing at least the surface with a functional group, in particular with a signalling molecule, an enzyme or a receptor molecule, such as an antibody. The receptor molecule may for instance be a receptor molecule for a component of interest, which is to be purified or detected, e.g. as part of a diagnostic test, making use of the particles of the present invention. Suitable functionalisation methods may be based on a method known in the art. In particular, the receptor molecule may be bound to the cross-linked polymer of which the particles are composed, via a reactive moiety in the residue X. An example of a reactive moiety in residue X is a carbodiimide group or a succinamide group.

If the microparticles for example comprise —OH and/or —COOH groups, for example in the X-moiety it is possible to functionalize such an —OH or —COOH group with a carbodiimide which may further react with a hydroxyl group of a target functional moiety to be coupled to the particles.

To couple a target functional moiety comprising an amide group N-hydroxysuccinimide (NHS) may be used. In particular NHS may be coupled to the microparticles if the microparticles comprise a polyalkylene glycol moiety, such as a PEG moiety. Such polyalkylene glycol moiety may in particular be the X residue or part thereof as presented in Formula II.

A target functional moiety may also comprise an —SH group, for example a cysteine residue which may be coupled to the microparticles by first reacting the microparticles with vinyl sulfone. In particular vinyl sulfone may be coupled to the microparticles if the microparticles comprise a polyalkylene glycol moiety, such as a PEG moiety. Such polyalkylene glycol moiety may in particular be the X group or part thereof as presented in Formula II. Various other coupling agents are known, (See Fisher et. al. Journal of Controlled release 111 (2006) 135-144 and Kasturi et. al. Journal of Controlled release 113 (2006) 261-270.

In principle microparticles may be prepared in a manner known in the art, provided that the polymers used in the prior art are (at least partially) replaced by the cross-linker (a) and that the reactive diluent (b) is present.

The weight to weight ratio of the reactive diluent (b) and cross-linker (a) may be 0 or more, usually at least 10:90, in particular at least 30:70 or at least 45:55. Preferably, the ratio is 90:10 or less, in particular 55:45 or less or 35:65 or less.

In addition to the cross-linker (a) and the reactive diluent (b), the microparticles of the present invention may further comprise one or more other compounds selected from the group of polymers and cross-linkable or polymerisable compounds. The polymers may in particular be polymers such as described above. The cross-linkable or polymerisable compounds may in particular be compounds selected from the group of acrylic compounds and other olefinically unsaturated compounds, for example, vinyl ether, allylether, allylurethane, fumarate, maleate, itaconate or unsaturated acrylate units. Suitable unsaturated acrylates are, for example, unsaturated urethaneacrylates, unsaturated polyesteracrylates, unsaturated epoxyacrylates and unsaturated polyetheracrylates.

The other polymers or polymerisable compounds may be used to adjust a property of the microparticles, for example to further tune the release profile of an active agent or to obtain a complete polymerization (i.e. no residual reactive unsaturated bonds that may be cytotoxic) or to narrow the size distribution of the microparticle. In case the microparticles are prepared from a combination of the cross-linker (a), the reactive diluent (b) and one or more other polymerisable compounds, cross-linked polymers may be formed, composed of cross-linker (a), reactive diluent (b) and the one or more other compounds.

The weight to weight ratio of the group of other polymers and polymerisable compounds to the total amount of cross-linker (a) and the reactive diluent (b) may be 0 or more. If another polymer or polymerisable compound is present, the weight to weight ratio of the group of other polymers and polymerisable compounds to the total amount of the cross-linker (a) and the reactive diluent (b) is usually at least 10:90, in particular at least 25:75 or at least 45:55. Preferably, the ratio is 90:10 or less, in particular 55:45 or less or 35:65 or less.

The microparticle is for example prepared comprising the steps of

selecting a reactive diluent (b) depending on the structure of a selected active agent (c) to be loaded into the microparticle

mixing cross-linker (a) with reactive diluent (b) and optionally a thermal initiator, a photoinitiator or a redox initiator;

making droplets comprising the reaction product and cross-linking the reaction product, resulting in the microparticle.

A microparticle loaded with active agents can for example be prepared comprising the steps of:

selecting a reactive diluent (b) depending on the structure of a selected active agent (c) to be loaded into the microparticle

mixing cross-linker (a) with reactive diluent (b) and optionally a thermal initiator, a photoinitiator or a redox initiator;

making droplets comprising the reaction product;

cross-linking the reaction product, resulting in the microparticle;

dissolving the active agent (c) in solvent (d);

immersing the microparticle with the solution of the active agent (c) in the solvent (d).

removal of the solvent (d) from the microparticle solution.

The solvent may be removed by solvent evaporation or by freeze drying.

Solvent (d) can be any liquid in which active agent (c) dissolves and which is not reactive towards active agent (c). Examples include alcohols, chlorinated solvents, tetrahydrofuran (THF), water, ethers, esters, phosphonated buffers, ketones, for example acetone, dimethylformamide (DMF), dimethylsulfoxide (DMSO), and N-methylpyrrolidone (NMP).

If a cross-linker according to Formula II is used, the microparticle is for example prepared by the steps of

reacting the multifunctional radically polymerisable compound X with an isocyanate represented by the Formula III.

O═C═N—R₆—C(R₃)═CHR₄ Formula III

wherein X, R₃, R₄ and R₆ are as defined herein above;

mixing the reaction product (represented by Formula II) with the reactive diluent (b)

forming droplets comprising the reaction product and the reactive diluent (b)

and cross-linking the reaction product.

An advantage of such method is its simplicity whereby the microparticle can be prepared starting from only two starting materials: a compound providing X and the compound of Formula III, especially for compounds of Formula III that are commercially available.

An alternative preparation route is via the reaction:

X+OCN—R₇—NCO+HO—R₈-A-C(═O)—C(R₃)═CH₂

wherein R₇ is an aliphatic, cycloaliphatic or aromatic group, wherein R₈ is an alkyl (C2-C4), wherein A is chosen from O or N and R₃ is as defined in Formula II.

Such alternative preparation method is advantageous for practical reasons, especially in terms of ease of commercially obtaining raw materials with various R-groups. Instead of an isocyanate also a thioisocyanate can be used.

The droplets are preferably formed by making an emulsion comprising the reaction product in a discontinuous phase. The compound of Formula II may be emulsified in for example water, an aqueous solution or another liquid or solvent. The stability of the emulsion may be enhanced by using known surfactant, for example triton X, polyethylene glycol or Tween 80. Using emulsion polymerisation is simple and is in particular suitable for a batch-process.

It is also possible to prepare the droplets making use of extrusion, spray drying or ink jet technology. Herein, a liquid comprising the reaction product is extruded or “jetted”, typically making use of a nozzle, into a suitable gas, e.g. air, nitrogen, a noble gas or the like, or into a non-solvent for the liquid and the reaction product. The size of the droplets can be controlled by the viscosity of the formulation, the use of a vibrating nozzle and/or a nozzle where a electrical filed is applied. By selecting a suitable temperature for the non-solvent or the gas and/or by applying another condition, e.g. radiation, cross-linking is accomplished, thereby forming the microparticles of the invention, e.g. as described in Espesito et al., Pharm. Dev. Technol 5(2); 267-278 or Ozeki et. al. Journal of controlled release 107 (2005) 387-394. Such process is in particular suitable to be carried out continuously, which may in particular be advantageous in case large volumes of the microparticles are to be prepared.

The reaction temperature is usually above the melting temperature of the cross-linker (a). It is also an option to dissolve the compound in a solvent, below or above the melting temperature of the compound. Besides allowing forming the droplets at a relatively low temperature, this may be useful in order to prepare porous particles. It is also possible to use a reactive solvent, for example a solvent that may react with the polymerising reagents, for instance a solvent that is a radically polymerisable monomer. In this way a fine tuning of the network density of the microparticle can be achieved. The temperature is generally below the boiling temperature of the liquid phase(s).

Cross-linking may be carried out in any suitable way known for cross-linking compounds comprising vinyl groups, in particular by thermal initiation (aided by a thermo initiator, such as a peroxide or an azo-initatior, e.g. azobisisobutylonitrile), by photo-initiation (aided by a photo-initiator such as a Norrish type I or II initiator), by redox-initiation (aided by a redox initiator), or any (other) mechanism that generates radicals making use of a chemical compound and/or electromagnetic radiation. Examples of suitable cross-linkers are trimethylolpropane trimethacrylate, diethylene glycol dimethacrylate or hydroxyethylacrylate.

In accordance with the invention it is possible to provide microparticles with one or more active agents with satisfactory encapsulation efficiency. Herein the encapsulation efficiency is defined as the amount of active agent in the particles after subjecting the loaded microparticles to one or more washing steps for 24 hours, divided by the amount of active agent used to load the microparticles, and can be determined for example by measuring the amount of active agent that is removed in the washing steps. Depending upon the loading conditions, an efficiency of at least about 20%, at least about 30%, at least about 40%, at least about 50%, at least about 75% or at least 90% or more is feasible.

The invention will now be illustrated by the following examples without being limited thereto.

### Materials and Methods

Dimethylaminoethyl methacrylate (DMAEMA), tetrahydrofurfuryl methacrylate (THFMA), 2-(Acetoacetoxy)ethyl methacrylate (AAEMA), 2-hydroxyethyl acrylate (HEA), phenoxyethyl acrylate (PhEA), Polyethyleneglycol methylether methacrylate (PEGMEA), ethyl acrylate (EA), 1,1,1-tris(hydroxymethyl)propane and Tin (II) 2-ethylhexanoate were purchased from Sigma-Aldrich. Polyvinyl alcohol (PVA) (88% hydr. M.W.=22.000) was purchased at Acros organics. Ebecryl 1040 was purchased from Cytec industries. Dimethylsulphoxide (DMSO), Tetrahydrofuran (THF), 1,4-dioxane and dichloromethane (DCM) were purchased from Merck. N-Hexane was purchased from VWR. Thiodiethylene bis[3-(3,5-di-tert-butyl-4-hydroxyphenyl)propionate] (Irganox 1035) and 2-hydroxy-2-methylpropiophenon (Darocure 1173) were purchased from Ciba Speciality Chemicals. D,L-lactide and Glycolide were purchased from Purac. L-lysinediisocyanate ethyl ester (OEt-LDI) was purchased from DSL Chemicals. L-lysinediisocyanate ethyl ester was vacuum distilled before use. 1,1,1-tris(hydroxymethyl)propane was recrystallized from ethyl acetate before use. The other chemicals were used as such.

Nuclear Magnetic Resonance (NMR) experiments were performed on a Varian Inova 300 spectrometer.

Infrared experiments were performed on a Perkin Elmer Spectrum One FT-IR Spectrometer.

(meth)Acrylate conversions measured were performed on a Perkin Elmer Spectrum One FTIR spectrometer equipped with a attenuated total reflection (ATR) accessory was used. Infrared spectra between 4000 and 650 cm⁻¹ were recorded averaging 20 scans with a spectral resolution of 4 cm⁻¹. The transmission spectra were transformed in absorption spectra. The peak height was determined at 1640 and 815 cm⁻¹ to measure double bond consumption.

Microparticles where prepared via mechanical agitation with an Ultra-turrax (Janke & Kunkel IKA Labortechnik model T25)

LST 200 Series Laser Diffraction Particle size analyzer (Beckman Coulter) was used to measure size distribution of the microparticles. The standard was UHMwPE (>50 μm).

A Leica DMLB microscope (magnitude×50 to ×400) was used to analyse the morphology of the microparticles.

Molar weight distributions were measured on a Waters GPC fitted with a Waters 2410 Refractive index detector and a Waters dual A absorbance UV-detector

### EXAMPLE 1

### Synthesis of (PLGA)₁₅₅₀(OH)₃

Glycolide (48.63 gram, 0.4189 mol) D,L-lactide (60.62 gram, 0.4206 mol), and 1,1,1-tris(hydroxymethyl)propane (10.43 gram, 0.07777 mol) were stirred together in a 500 ml reaction flask under nitrogen and heated up to 150° C. A Catalyst solution was made by dissolving tin(II) 2-ethyl hexanoate (189 mg) (0.05% (m/m) with respect to the total weight of reactants) in 1 ml n-hexane. This solution was added to the reaction mixture at 150° C. This was stirred at 150° C. for 18 hours upon the reaction was complete as indicated by NMR. ¹H-NMR (300 MHz, CDCl₃, 22° C., TMS): δ (ppm)=5.3-5.1 (8.6H, m, CH), 4.8-4.6 (17H, m, CO—CH₂—O), 4.3-4.0 (10.5H, m, C—CH₂+CH—OH+CO—CH₂—OH), 1.8-1.2 (22.4H, m, CH₃—CH₂+CH₂—CH₂—CH₂—CH₂) 0.9 (3H, m CH₃—CH₂).

### EXAMPLE 2

### Synthesis of OEt-LDI-HEA

L-Lysine diisocyanate ethyl ester (OEt-LDI) (247.17 gram, 1.0926 mol), 450 mg (0.12 wt. % based on total weight) of Irganox 1035 and 180 mg (0.048% (m/m) with respect to the total weight of reactants) of tin(II) 2-ethyl hexanoate were stirred together in a 100-ml reaction flask under dry air at room temperature. 126.54 g (1.0898 mol) 2-hydroxyethyl acrylate was added drop wise in 10 min. The reaction mixture was and stirred for 18 hours at 40° C. upon the reaction was complete as indicated by NMR. ¹H-NMR (300 MHz, CDCl₃, 22° C., TMS): δ (ppm)=6.4 (H, m, CH, Cis acrylate), 6.2 (H, m, CH—C═O, acrylate), 5.9 (H, m, CH, Trans, acrylate), 5.4 (H, broad, NH—CH), 4.8 (H, broad, NH—CH₂), 4.4-4.2 (7H, m, O—CH₂—CH₃+O—CH₂—CH₂—O+O—CH₂—CH₂—O+CH—NH), 4.0 (H, m, CH—NCO), 3.4 (2H, m, CH₂—NCO), 3.2 (2H, m, CH₂—NH), 1.9-1.3 (8H, m, CH₂—CH₂CH₂—CH₂+O—CH₂—CH₃).

### EXAMPLE 3

### Synthesis of (PLGA)₁₅₅₀(OEt-LDI-HEA)₃ 5317-25

(PLGA)₁₅₅₀(OH)₃ (119.68 gram, 0.09832 mol), 304 mg (0.19 wt. % based on total weight) of Irganox 1035, 121 mg (0.08% (m/m) with respect to the total weight of reactants) of tin(II) 2-ethyl hexanoate and 100 ml THF were stirred together in a 100 ml reaction flask under dry air. 48.41 g (0.1414 mol) OEt-LDI-HEA was added drop wise in 30 min. The reaction mixture was and stirred for 18 hours at 30° C. upon the reaction was complete as indicated by IR and NMR. THF was removed on a rotation evaporator. ¹H-NMR (300 MHz, CDCl₃, 22° C., TMS): δ (ppm)=6.4 (2H, m, CH, Cis acrylate), 6.2 (2H, m, CH—C═O, acrylate), 5.9 (2H, m, CH, Trans, acrylate), 5.6 (2H, broad, NH—CH), 5.4 (2H, broad, NH—CH₂), 5.3-5.1 (8.6H, m, CH), 4.8-4.6 (17H, m, CO—CH₂—O, 4.4-4.0 (26H, m, O—CH₂—CH₂—O+C—CH₂+CH—NH+O—CH₂—CH₃), 3.1 (4H, m, CH₂—NH), 1.9-1.2 (54.7H, m, CH—CH₃+CH₂—CH₃+CH₂—CH₂—CH₂—CH₂+O—CH₂—CH₃), 0.9 (3H, m CH₃—CH₂).

### EXAMPLE 4

### Microparticles (PLGA)₁₅₅₀(OEt-LDI-HEA)₃ with Ebecryl 1040

A preformulation of 7.1807 g (PLGA)₁₅₅₀(OEt-LDI-HEA)₃, 3.0133 g Ebecryl 1040 and 0.1009 Darocure 1173 was prepared. Also a 1% (m/m) PVA stock solution of 10.49 g PVA in 1001.2 g demineralized water was prepared. A formulation of 1.452 g of preformulation and 0.382 g DCM was prepared.

A mixture of 1.361 g of formulation and 30.024 g PVA stock solution was agitated at 8000 rpm in a 50 ml beaker at room temperature for 1 minute. Now polymerization was allowed to proceed for 30 min under UV light (Macam Flexicure controller, D-bulb, 400 mW/s/cm2). Afterwards double bond consumption was checked: >98% (FT-IR, 1640 cm⁻¹ and 810 cm¹). Now microparticles were washed via centrifuging with 6 times 10 ml demineralised water, the supernatant was decanted off.

Microparticles where dried via freeze drying for 70 hours.

### EXAMPLE 5

### Microparticles (PLGA)₁₅₅₀(OEt-LDI-HEA)₃ with EA

A preformulation of 7.1207 g (PLGA)₁₅₅₀(OEt-LDI-HEA)₃, 2.9786 g EA and 0.1029 g Darocure 1173 was prepared. Also a 1% (m/m) PVA stock solution of 10.49 g PVA in 1001.2 g demineralized water was prepared. A formulation of 1.4723 g of preformulation and 0.4050 g DCM was prepared.

A mixture of 1.516 g of formulation and 29.97 g PVA stock solution was agitated at 8000 rpm in a 50 ml beaker at room temperature for 1 minute. Now polymerization was allowed to proceed for 30 min under UV light (Macam Flexicure controller, D-bulb, 400 mW/s/cm2). Afterwards double bond consumption was checked: >98% (FT-IR, 1640 cm⁻¹ and 810 cm¹). Now microparticles were washed via centrifuging with 6 times 10 ml demineralised water, the supernatant was decanted off. Microparticles where dried via freeze drying for 70 hours

### EXAMPLE 6

### Microparticles (PLGA)₁₅₅₀(OEt-LDI-HEA)₃ with PEGMEA

A preformulation of 7.2377 g (PLGA)₁₅₅₀(OEt-LDI-HEA)₃, 3.0378 g PEGMEA and 0.1054 g Darocure 1173 was prepared. Also a 1% (m/m) PVA stock solution of 10.49 g PVA in 1001.2 g demineralized water was prepared. A formulation of 1.4571 g of preformulation and 0.368 g DCM was prepared.

A mixture of 1.415 g of formulation and 20.211 g PVA stock solution was agitated at 8000 rpm in a 50 ml beaker at room temperature for 1 minute. Now polymerization was allowed to proceed for 30 min under UV light (Macam Flexicure controller, D-bulb, 400 mW/s/cm2). Afterwards double bond consumption was checked: >98% (FT-IR, 1640 cm⁻¹ and 810 cm¹). Now microparticles were washed via centrifuging with 6 times 10 ml demineralised water, the supernatant was decanted off.

Microparticles where dried via freeze drying for 70 hours

### EXAMPLE 7

### Microparticles (PLGA)₁₅₅₀(OEt-LDI-HEA)₃ with PhEA

A preformulation of 7.2392 g (PLGA)₁₅₅₀(OEt-LDI-HEA)₃, 3.1438 g PhEA and 0.1197 g Darocure 1173 was prepared. Also a 1% (m/m) PVA stock solution of 10.49 g PVA in 1001.2 g demineralized water was prepared. A formulation of 1.4816 g of preformulation and 0.569 g DCM was prepared.

A mixture of 1.794 g of formulation and 30.336 g PVA stock solution was agitated at 8000 rpm in a 50 ml beaker at room temperature for 1 minute. Now polymerization was allowed to proceed for 30 min under UV light (Macam Flexicure controller, D-bulb, 400 mW/s/cm2). Afterwards double bond consumption was checked: >98% (FT-IR, 1640 cm⁻¹ and 810 cm¹). Now microparticles were washed via centrifuging with 6 times 10 ml demineralised water, the supernatant was decanted off.

Microparticles where dried via freeze drying for 70 hours.

### EXAMPLE 8

### Microparticles (PLGA)₁₅₅₀(OEt-LDI-HEA)₃ with HEA

A preformulation of 6.9182 g (PLGA)₁₅₅₀(OEt-LDI-HEA)₃, 2.9942 g HEA and 0.1055 g Darocure 1173 was prepared. Also a 1% (m/m) PVA stock solution of 10.49 g PVA in 1001.2 g demineralized water was prepared. A formulation of 1.5152 g of preformulation and 0.399 g DCM was prepared.

A mixture of 1.462 g of formulation and 30.02 g PVA stock solution was agitated at 8000 rpm in a 50 ml beaker at room temperature for 1 minute. Now polymerization was allowed to proceed for 30 min under UV light (Macam Flexicure controller, D-bulb, 400 mW/s/cm2). Afterwards double bond consumption was checked: >98% (FT-IR, 1640 cm⁻¹ and 810 cm¹). Now microparticles were washed via centrifuging with 6 times 10 ml demineralised water, the supernatant was decanted off.

Microparticles where dried via freeze drying for 70 hours.

### EXAMPLE 9

### Microparticles (PLGA)₁₅₅₀(OEt-LDI-HEA)₃ with AAEMA

A preformulation of 7.1248 g (PLGA)₁₅₅₀(OEt-LDI-HEA)₃, 3.0106 g AAEMA and 0.0987 g Darocure 1173 was prepared. Also a 1% (m/m) PVA stock solution of 10.49 g PVA in 1001.2 g demineralized water was prepared. A formulation of 1.4763 g of preformulation and 0.366 g DCM was prepared.

A mixture of 1.215 g of formulation and 30.03 g PVA stock solution was agitated at 8000 rpm in a 50 ml beaker at room temperature for 1 minute. Now polymerization was allowed to proceed for 30 min under UV light (Macam Flexicure controller, D-bulb, 400 mW/s/cm2). Afterwards double bond consumption was checked: >98% (FT-IR, 1640 cm⁻¹ and 810 cm¹). Now microparticles were washed via centrifuging with 6 times 10 ml demineralised water, the supernatant was decanted off.

Microparticles where dried via freeze drying for 70 hours.

### EXAMPLE 10

### Microparticles (PLGA)₁₅₅₀(OEt-LDI-HEA)₃ with THFMA

A preformulation of 6.8471 g (PLGA)₁₅₅₀(OEt-LDI-HEA)₃, 3.0060 g THFMA and 0.1028 g Darocure 1173 was prepared. Also a 1% (m/m) PVA stock solution of 10.49 g PVA in 1001.2 g demineralized water was prepared. A formulation of 1.5165 g of preformulation and 0.3968 g DCM was prepared.

A mixture of 1.59 g of formulation and 30.07 g PVA stock solution was agitated at 8000 rpm in a 50 ml beaker at room temperature for 1 minute. Now polymerization was allowed to proceed for 30 min under UV light (Macam Flexicure controller, D-bulb, 400 mW/s/cm2). Afterwards double bond consumption was checked: >98% (FT-IR, 1640 cm⁻¹ and 810 cm¹). Now microparticles were washed via centrifuging with 6 times 10 ml demineralised water, the supernatant was decanted off.

Microparticles where dried via freeze drying for 70 hours.

### EXAMPLE 11

### Microparticles (PLGA)₁₅₅₀(OEt-LDI-HEA)₃ with DMAEMA

A preformulation of 7.5213 g (PLGA)₁₅₅₀(OEt-LDI-HEA)₃, 3.0016 g DMAEMA and 0.1018 g Darocure 1173 was prepared. Also a 1% (m/m) PVA stock solution of 10.49 g PVA in 1001.2 g demineralized water was prepared. A formulation of 1.4631 g of preformulation and 0.3648 g DCM was prepared.

A mixture of 1.558 g of formulation and 30.05 g PVA stock solution was agitated at 8000 rpm in a 50 ml beaker at room temperature for 1 minute. Now polymerization was allowed to proceed for 30 min under UV light (Macam Flexicure controller, D-bulb, 400 mW/s/cm2). Afterwards double bond consumption was checked: >98% (FT-IR, 1640 cm⁻¹ and 810 cm¹). Now microparticles were washed via centrifuging with 6 times 10 ml demineralised water, the supernatant was decanted off.

Microparticles where dried via freeze drying for 70 hours.

### Comparative Experiment A: Microparticles (PLGA)₁₅₅₀(OEt-LDI-HEA)₃

A preformulation of 7.6019 g (PLGA)₁₅₅₀(OEt-LDI-HEA)₃, 1.9172 g DCM and 0.0743 Darocure 1173 was prepared. Also a 1% (m/m) PVA stock solution of 10.49 g PVA in 1001.2 g demineralized water was prepared. A formulation of 1.25 g of preformulation and 0.78 g DCM was prepared.

A mixture of 1.741 g of formulation and 19.992 g PVA stock solution was agitated at 8000 rpm in a 50 ml beaker at room temperature for 1 minute. Now polymerization was allowed to proceed for 30 min under UV light (Macam Flexicure controller, D-bulb, 400 mW/s/cm2). Afterwards double bond consumption was checked: >98% (FT-IR, 1640 cm⁻¹ and 810 cm¹). Now microparticles were washed via centrifuging with 6 times 10 ml demineralised water, the supernatant was decanted off.

Microparticles where dried via freeze drying for 70 hours.

### EXAMPLE 12

### Loading of Microparticles with a Drug Via Solvent Evaporation

In table 2 a stock solution of dexamethason in THF was prepared. From this solution an amount was added to a centrifuge tube containing 30 (±2) mg microparticles.

Afterwards THF was evaporated from the centrifuge tubes by putting these on a roller bench for 18 hours.

TABLE 2
loading amounts of dexamethasone

| | microspheres | stock | Dexamethasone | Dexamethasone |
|---|---|---|---|---|
| reactive diluent | (mg) | (mg) | (ug) | (%) |
| none | 32.24 | 86.83 | 1486 | 4.41 |
| none | 28.90 | 88.02 | 1507 | 4.96 |
| none | 30.27 | 88.43 | 1514 | 4.76 |
| HEA | 30.75 | 88.43 | 1514 | 4.69 |
| HEA | 30.91 | 87.34 | 1495 | 4.61 |
| HEA | 29.83 | 88.23 | 1510 | 4.82 |
| PEGMEA | 30.90 | 88.18 | 1509 | 4.66 |
| PEGMEA | 30.57 | 92.26 | 1579 | 4.91 |
| PEGMEA | 30.44 | 89.88 | 1539 | 4.81 |
| EA | 31.04 | 88.03 | 1507 | 4.63 |
| EA | 30.09 | 86.00 | 1472 | 4.66 |
| EA | 30.13 | 87.55 | 1499 | 4.74 |
| Ebecryl 1040 | 29.47 | 88.05 | 1507 | 4.87 |
| Ebecryl 1040 | 29.91 | 87.63 | 1500 | 4.78 |
| Ebecryl 1040 | 32.52 | 87.79 | 1503 | 4.42 |
| THFFMA | 29.31 | 88.41 | 1513 | 4.91 |
| THFFMA | 29.54 | 88.42 | 1514 | 4.87 |
| THFFMA | 29.96 | 88.51 | 1515 | 4.81 |
| DMAEMA | 30.92 | 87.23 | 1493 | 4.61 |
| DMAEMA | 30.46 | 87.75 | 1502 | 4.70 |
| DMAEMA | 30.73 | 88.57 | 1516 | 4.70 |
| PhEA | 29.20 | 87.98 | 1506 | 4.90 |
| PhEA | 31.45 | 88.21 | 1510 | 4.58 |
| PhEA | 29.92 | 88.68 | 1518 | 4.83 |
| AAEMA | 28.95 | 86.74 | 1485 | 4.88 |
| AAEMA | 30.52 | 89.52 | 1532 | 4.78 |
| AAEMA | 30.91 | 87.16 | 1492 | 4.60 |

### EXAMPLE 13

### Loading of Microparticles with a Drug Via Freeze Drying

In table 3 a stock solution of dexamethason in 1,4-dioxane was prepared. From this solution an amount was added to a centrifuge tube containing 30 (±2) mg microparticles.

Afterwards 1,4-dioxane was evaporated from the centrifuge tubes by putting these in a freeze dryer for 18 hours.

TABLE 3
loading amounts of dexamethasone

| Reactive | microspheres | stock | Dexamethasone | Dexamethasone |
|---|---|---|---|---|
| diluent | (mg) | (mg) | (ug) | (%) |
| none | 29.80 | 103.21 | 1642 | 5.22 |
| none | 31.09 | 101.94 | 1621 | 4.96 |
| none | 30.97 | 103.34 | 1644 | 5.04 |
| HEA | 30.99 | 99.72 | 1586 | 4.87 |
| HEA | 30.60 | 100.89 | 1605 | 4.98 |
| HEA | 31.03 | 99.21 | 1578 | 4.84 |
| PEGMEA | 31.28 | 101.81 | 1619 | 4.92 |
| PEGMEA | 31.09 | 101.57 | 1616 | 4.94 |
| EA | 31.65 | 102.00 | 1622 | 4.88 |
| EA | 31.18 | 100.90 | 1605 | 4.90 |
| EA | 31.46 | 101.18 | 1609 | 4.87 |
| Ebecryl 1040 | 28.95 | 99.14 | 1577 | 5.17 |
| Ebecryl 1040 | 29.79 | 100.00 | 1591 | 5.07 |
| Ebecryl 1040 | 30.58 | 99.53 | 1583 | 4.92 |
| THFFMA | 31.00 | 98.17 | 1561 | 4.80 |
| THFFMA | 31.04 | 100.15 | 1593 | 4.88 |
| THFFMA | 31.60 | 100.76 | 1603 | 4.83 |
| DMAEMA | 30.51 | 99.65 | 1585 | 4.94 |
| DMAEMA | 30.86 | 98.91 | 1573 | 4.85 |
| DMAEMA | 30.07 | 94.91 | 1510 | 4.78 |
| PhEA | 29.73 | 99.67 | 1585 | 5.06 |
| PhEA | 30.21 | 100.20 | 1594 | 5.01 |
| PhEA | 31.25 | 99.49 | 1582 | 4.82 |
| AAEMA | 30.87 | 97.28 | 1547 | 4.77 |
| AAEMA | 29.77 | 96.65 | 1537 | 4.91 |
| AAEMA | 29.48 | 99.99 | 1590 | 5.12 |

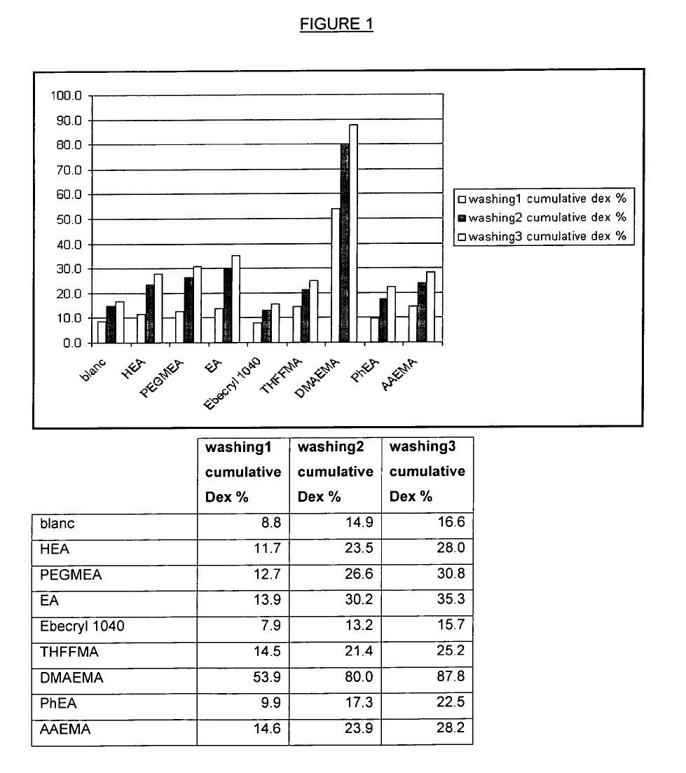

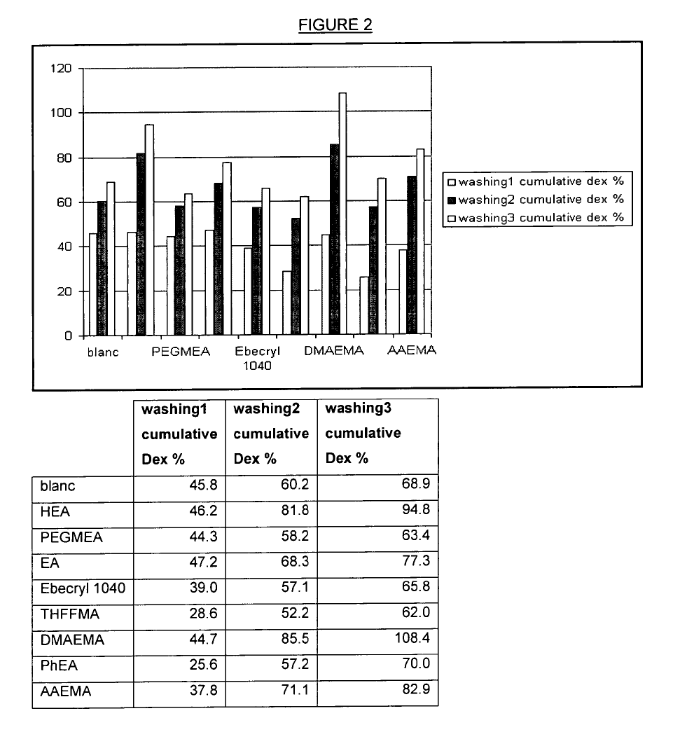

FIGS. 1 and 2 show the encapsulation efficiency which was determined by measuring the amount of active agent that is removed in the washing steps. The figures moreover show the ability to tune the release in case that a reactive diluent is present if compared with the blanc.

FIG. 1 shows the result of loading the microparticles via solvent evaporation.

FIG. 2 which is the result of loading the microspheres via freeze drying shows a faster or slower release when compared to the blanc.

1. Microparticle comprising a cross-linked polymer comprising
(a) a cross-linker comprising two or more radically polymerizable groups, preferably selected from the group consisting of alkenes, sulfhydryl (SH), thioic, unsaturated esters, unsaturated urethanes, unsaturated ethers, and unsaturated amides;
(b) a monofunctional reactive diluent comprising maximum one unsaturated C—C bond represented by the formula
R₀—C(R₁)═CHR₂ Formula I
wherein
R₀ is chosen depending on the structure of a selected active agent (c) to be loaded into the microparticle and is chosen to have a structure that when combined with the other components of the microparticle provides a higher affinity of the selected active agent (c) for the microparticle;
each R₁ is chosen from hydrogen and substituted and unsubstituted, aliphatic, cycloaliphatic and aromatic hydrocarbon groups which groups optionally contain one or more moieties selected from the group of ester moieties, ether moieties, thioester moieties, thioether moieties, carbamate moieties, thiocarbamate moieties, amide moieties and other moieties comprising one or more heteroatoms, in particular one or more heteroatoms selected from S, O, P and N.
each R₂ is chosen from hydrogen, —COOCH₃, —COOC₂H₅, —COOC₃H₇, and —COOC₄H₉.

2. Microparticle according to claim 1, wherein R₀ is a linear, (hyper)branched or cyclic functional group optionally possessing a heteroatom chosen from the group consisting of O, N, S, or P.

3. Microparticle according to claim 2, wherein R₀ is a linear or (hyper)branched functional group comprising amine, amide, carbamate, urea, thiol, hydroxyl, carboxyl, ester, ether, thioester, thioester carbonate, phosphate, posphite, sulphate, sulphoxide and/or sulphone groups.

4. Microparticle according to claim 2, wherein R₀ is a cyclic functional group chosen from the group consisting of 5-membered ring phosphate, 6-membered ring phosphate, 5-membered ring phosphite, 6-membered ring phosphite, 4-membered ring lacton, 5-membered ring lacton, 6-membered ring lacton, 5-membered ring carbonate, 6-membered ring carbonate, 5-membered ring sulphate, 6-membered ring sulphate, 5 ring sulphoxide, 6-membered ring sulphoxide, 6-membered ring amide, 5-membered ring urethane, 6-membered ring urethane, 7-membered ring urethane, 5-membered ring urea, 6-membered ring urea, and 7-membered ring urea.

5. Microparticles according to claim 1 wherein the cross-linker (a) comprises two or more —CR₃═CHR₄ groups wherein
each R₃ is independently chosen from hydrogen and substituted and unsubstituted, aliphatic, cycloaliphatic and aromatic hydrocarbon groups which groups optionally contain one or more moieties selected from the group of ester moieties, ether moieties, thioester moieties, thioether moieties, carbamate moieties, thiocarbamate moieties, amide moieties and other moieties comprising one or more heteroatoms, in particular one or more heteroatoms selected from S, O, P and N.
each R₄ is chosen from hydrogen, —COOCH₃, —COOC₂H₅, —COOC₃H₇, —COOC₄H₉,

6. Microparticles according to claim 1 wherein the cross-linker (a) is represented by the formula
X—[Y—C(═Z)—N(R₅)—R₆—C(R₃)═CR₄]ₙ Formula II
wherein
X is a residue of a multifunctional radically polymerisable compound (having at least a functionality equal to n);
each Y independently is optionally present, and—if present—each Y independently represents a moiety selected from the group of O, S and NR₅;
each Z is independently chosen from O and S;
each R₃ and R₄ are as defined in claim 5;
each R₅ is independently chosen from the group of hydrogen and substituted and unsubstituted, aliphatic, cycloaliphatic and aromatic hydrocarbon groups which groups optionally contain one or more moieties selected from the group of ester moieties, ether moieties, thioester moieties, thioether moieties, carbamate moieties, thiocarbamate moieties, amide moieties and other moieties comprising one or more heteroatoms, in particular one or more heteroatoms selected from S, O, P and N.
each R₆ is independently chosen from the group of substituted and unsubstituted, aliphatic, cycloaliphatic and aromatic hydrocarbon groups which groups optionally contain one or more moieties selected from the group of ester moieties, ether moieties, thioester moieties, thioether moieties, carbamate moieties, thiocarbamate moieties, amide moieties and other moieties comprising one or more heteroatoms, in particular one or more heteroatoms selected from S, O, P and N; and
n is at least 2.

7. Microparticle according to claim 6, wherein X is the residue of a OH, —NH₂, —RNH or —SH multifunctional polymer or oligomer.

8. Microparticle according to claim 6 wherein X is selected from a biostable or biodegradable polymer or oligomer.

9. Microparticle according to claim 8, wherein X is selected from an aliphatic polyester, aliphatic polythioester, aliphatic polythioether, aliphatic polyether or polypeptide.

10. Microparticle according to claim 6 wherein R₅ is hydrogen or an alkyl group.

11. Microparticle according to claim 6 wherein R₆ comprises 2-20 carbon atoms, preferably 2-14 carbon atoms.

12. Microparticle according to claim 6 wherein R₃ is hydrogen or comprises 1-6 carbon atoms.

13. Microparticle according to claim 1, wherein the average diameter is in the range of 10 nm to 1000 μm, preferably in the range of 1-100 μm.

14. Microparticle according to claim 1 wherein the microparticles are provided with a structure comprising an inner core and an outer shell.

15. Microparticle according to claim 1 comprising one or more active agents (c).

16. Microparticle according to claim 15, wherein the active agent (c) is selected from the group of nutrients, pharmaceuticals, proteins and peptides, vaccines, genetic materials, oligonucleotides, diagnostic agents or imaging agents.

17. Microparticle according claim 1, wherein the cross-linked polymer is a carbamate, thiocarbamate, ureyl or amide copolymer.

18. Method for preparing a microparticle according to claim 1 comprising the steps of
selecting a reactive diluent (b) depending on the structure of a selected active agent (c) to be loaded into the microparticle
mixing cross-linker (a) with reactive diluent (b) and optionally a thermal initiator, a photoinitiator or a redox initiator;
making droplets comprising the reaction product;
and cross-linking the reaction product, resulting in the microparticle.

19. Method for preparing a microparticle according to claim 18 loaded with one or more reactive agents (c) comprising the steps of:
dissolving the active agent (c) in a solvent (d);
immersing the microparticle with the solution of the active agent (c) in the solvent (d)
removal of the solvent from the microparticle solution.

20. Method according to claim 19 whereby the removal of the solvent is achieved by solvent evaporation or freeze drying.

21. Microparticle according to claim 1 for medical use.

22. Use of a microparticle according to claim 1 for the manufacturing of a medicament for treatment in dermatology, vascular, orthopedics, ophthalmic, spinal, intestinal, pulmonary, nasal or auricular applications.

23. Microparticle according to claim 1 for use as a delivery system for an active agent.

24. Use of the microparticle according to claim 1 in suspensions, capsules, tubes, pellets, (rapid prototyped) scaffolds, coatings, patches, composite materials or plasters or (in situ forming) gels.
